(19) United States
(12) Patent Application Publication
(10) Pub. No.: US 2002/0098211 A1
(43) Pub. Date: Jul. 25, 2002

(54) ANTIMICROBIAL AGENT AND USES THEREOF, IN PARTICULAR IN COSMETICS AND DERMATOLOGY

(76) Inventors: SYLVIE CUPFERMAN, PARIS (FR); CATHERINE MARION, SCEAUX (FR)

Correspondence Address: OBLON SPIVAK MCCLELLAND MAIER & NEUSTADT, FOURTH FLOOR, 1755 JEFFERSON DAVIS HIGHWAY, ARLINGTON, VA, 22202

(21) Appl. No.: 09/437,095

(22) Filed: Nov. 9, 1999

(30) Foreign Application Priority Data
Nov. 9, 1998 (FR) 9814077

(51) Int. Cl. A61K 6/00; A61K 7/00

(52) U.S. Cl. 424/401

(57) ABSTRACT

The present invention relates to a novel antimicrobial agent, which withstands or allows the control of microbial degradation, comprising (I) at least one polyol and (II) 2-hydroxy-4-(1-methylethyl)cyclohepta-2 4 6-trier-1-one or sodium capryl lactyl lactylate, and to compositions, in particular cosmetic, dermatological, pharmaceutical or food compositions, containing such a agent in a physiologically acceptable support.

### BACKGROUND OF THE INVENTION

1. Field of the Invention

The present invention relates to a novel antimicrobial agent comprising (I) at least one polyol in combination and (II) 2-hydroxy-4-(1-methylethyl)cyclohepta-2,4,6-trien-1-one or sodium capryl lactyl lactylate. Additionally, the present invention relates to compositions, in particular cosmetic, dermatological, pharmaceutical or food compositions, containing the antimicrobial agent in a physiologically acceptable support.

It is common practice to introduce chemical preserving agents into cosmetic or dermatological compositions in order to control the growth of microorganisms in these compositions, which would otherwise make them unsuitable for use. In particular, to protect against microorganisms that are able to grow in the composition and which the user might introduce into the composition by handling, for example, when the user takes products from a jar with the fingers.

Chemical preserving agents commonly used are, in particular, parabens or formaldehyde donors. These preserving agents have the drawback of irritations and/or allergies, and in particular, effects to sensitive skin. The use of certain alcohols or polyols, such as ethanol or propylene glycol, may also irritate or illicit allergies to the user. This is particularly true when the alcohols or polyols are present in relatively large proportions, i.e. in amounts exceeding 20% by weight relative to the composition as a whole.

FR-A-2,747,572 discloses the use of 1,2-alkanediols to inactivate skin microorganisms. However, these compounds at the concentrations usually used do not prevent the growth of a large number and/or variety of microorganisms. Furthermore, to increase the compound's efficacy it is necessary to introduce them into amounts whereby skin tolerance and levels of adverse side effects are no longer satisfactory in the product.

In addition, FR-A-2,729,050 discloses the combination of a C₂-C₈ polyol with at least one mono (C₃-C₉) alkyl or (C₃-C₉) alkenyl glyceryl ether and not more than 15% of aliphatic monoalcohol(s). However, the amount of polyols required to obtain broad-spectrum antimicrobial activity is incompatible with the cosmetic formulations as a whole and, can result in skin irritations.

It is thus observed that there is a need for antimicrobial agents which are at least as effective in their action as the compounds of the prior art, but which do not have their drawbacks.

Thus, after considerable research conducted in this matter, the Applicant has now discovered, surprisingly and unexpectedly, that a antimicrobial agent comprising a specific polyol combined with 2-hydroxy-4-(1-methylethyl)cyclohepta-2,4,6-trien-1-one or sodium capryl lactyl lactylate has high and synergistic antimicrobial action, thereby preserving compositions into which it is introduced.

This combination is advantageous since it does not result in any intolerance and/or redness when a cosmetic or dermatological composition comprising this antimicrobial agent is applied to the skin. Furthermore, the antimicrobial agent has an antimicrobial activity which is at least as effective as that of the compounds of the prior art.

The discovery of these novel antimicrobial agents forms the basis of the present invention.

### SUMMARY OF THE INVENTION

According to the invention, the term “antimicrobial agent” means a composition which withstands or allows the control of microbial degradation and thus has antibacterial activity and/or antifungal activity and/or antiviral activity.

A subject of the present invention is thus an antimicrobial agent, where it comprises (I) at least one polyol chosen from polyols comprising from 4 to 8 carbon atoms, mono (C₃-C₉) alkyl or (C₃-C₉) alkenyl glyceryl ethers; and (II) 2-hydroxy-4 (1-methylethyl)cyclohepta-2,4,6-trien-1-one.

Among the polyols comprising from 4 to 8 carbon atoms, the ones preferably used are those comprising from 5 to 8 carbon atoms, such as 1,2-octanediol, 1,2-pentanediol, isoprene glycol and sorbitol. Mono (C₃-C₉) alkyl or (C₃-Cg) alkenyl glyceryl ethers may be, for-example, octoxyglycerol (or 3-[(2-ethylhexyl)oxy]1,2-propanediol), 3-[(heptyl)oxy]-1,2-propanediol, 3-[(octyl)oxy]-1,2-propanediol, 3-[(allyl)oxy]-1,2-propanediol, or a mixture thereof. 1,2-octanediol, 1,2-pentanediol and/or octoxyglycerol are preferably used.

2-Hydroxy-4-(1-methylethyl)cyclohepta-2,4,6-trien-1-one (CTFA name: Hinokitiol) can be synthesized or can be obtained from plants and, for example, from oil extracted from trees, such as hiba oil or cedarwood oil. It is also possible to use 2-hydroxy-4-(1-methylethyl)cyclohepta-2,4,6-trien-1-one salts, and in particular the alkali metal (sodium or potassium) or alkaline-earth metal (magnesium) salts or salts of organic bases (diethanolamine).

A subject of the present invention is also an antimicrobial agent, where it comprises (I) at least one polyol chosen from 1,2-octanediol, 1,2-pentanediol, sorbitol and mono(C₃-C₉) alkyl or (C₃-C₉) alkenyl glyceryl ethers; and (II) sodium capryl lactyl lactylate.

Sodium capryl lactyl lactylate (CTFA name: sodium capryl lactylate) is a sodium salt of the caprylic ester of lactyl lactate.

The mono (C₃-C₉) alkyl or (C₃-C₉) alkenyl glyceryl ethers can be those mentioned above.

Another subject of the invention is the use of the antimicrobial agents defined above in a cosmetic or dermatological composition.

A subject of the present invention is also a composition comprising, in a physiologically acceptable support, at least one antimicrobial agent as defined above.

### DETAILED DESCRIPTION OF THE INVENTION

In the antimicrobial agents mentioned above, the weight ratio of the polyol (I) relative to the compound (II) can vary within a wide range. It preferably ranges from 0.1 to 20,000, more particularly from 0.25 to 100. These ranges include all specific values and subranges there between including weight ratios of 0.5, 1, 5, 10, 20, 30, 50, 60, 75, 90, 200, 500, 1000, 1500, 2000, 5000, 7500, 10000, 12500, 15000, and 17500.

The synergistic antimicrobial agents of the invention have the advantage of being effective on different microorganisms, and in particular on Enterococcus faecalis and Candida albicans. In addition, the polyol can be present in a composition in smaller amounts than when it is used alone.

These antimicrobial agents are more particularly intended for cosmetic, pharmaceutical, dermatological, or food use. When they are intended for topical application, such as cosmetic or dermatological application, they then comprise a physiologically acceptable support, i.e. a support which is compatible with the skin, the hair and/or mucous membranes.

According to one preferred embodiment of the invention, the composition according to the invention is free of conventional preserving agents.

The compositions comprising an antimicrobial system of the invention can contain, for example, from 0.01 to 20% and preferably from 0.1 to 5% by weight of polyol(s) (I) relative to the total weight of the composition. These ranges include all specific values and subranges there between including 0.05, 0.1, 0.5, 1, 3, 6, 9, 13, 15, and 18% by weight. The compositions comprising an antimicrobial system of the invention can contain, for example from 0.001 to 10% and better still from 0.01 to 5% by weight of compound(s) (II) relative to the total weight of the composition. These ranges include all specific values and subranges there between including 0.005, 0.01, 0.05, 0.1, 0.5, 1, 3, 6, and 9% by weight.

In the case of a cosmetic or dermatological application, the compositions of the invention can also comprise cosmetic and/or dermatological adjuvants conventionally used, such as fatty substances, organic solvents, ionic or nonionic thickeners, softeners, antioxidants, pacifiers, stabilizers, antifoaming agents, moisturizers, vitamins, fragrances, ionic or nonionic emulsifiers, fillers, sequestering agents, electrolytes dyes or any other ingredient usually used in cosmetics or dermatology. The amounts of the various adjuvants in the compositions according to the invention are those used conventionally in the fields under consideration. The nature of the adjuvants and their amounts must be such that they do not modify the properties of the compositions according to the invention. Other adjuvants that may be used in the present composition are disclosed in Kirk-Othmer Encyclopedia of Chemical Technology, Volume I, Fourth Edition, 1993, pages 572-619, incorporated herein by reference.

The compositions according to the invention can be in the form of a solution, a suspension or a dispersion in solvents such as water or fatty substances, in the form of a vesicle dispersion or alternatively in the form of a water-in-oil (W/O), oil-in-water (O/W) or multiple emulsion, such as a cream of a milk, or in the form of an ointment, a gel, a solid tube, an aerosol mousse or a spray.

When the composition is an emulsion, the proportion of the fatty phase can range from 5 to 80% by weight and preferably from 5 to 50% by weight relative to the total weight of the composition. These ranges include all specific values and subranges there between including 9, 13, 19, 26, 31, 42, 49, 55, 66, 72, and 78% by weight. The oils, waxes, emulsifiers and co-emulsifiers used in the composition in the emulsion form are chosen from those used conventionally in cosmetics and dermatology. The emulsifier and the co-emulsifier are present in the composition in a proportion preferably ranging from 0.3 to 30% by weight and better still from 0.5 to 20% by weight relative to the total weight of the composition. These ranges include all specific values and subranges there between including 0.9, 1.5, 4, 7, 14, 19, and 26% by weight. The emulsion can also contain lipid vesicles.

Fatty substances which can be used, for example, are oils of plant origin, volatile silicone oils (cyclomethicone) or non-volatile silicone oils, mineral oils (petroleum jelly, isoparaffin), synthetic oils and in particular fatty esters, fatty alcohols (cetyl alcohol), fatty acids and waxes. Other fatty substances that may be used in the present composition are disclosed in Kirk-Othmer Encyclopedia of Chemical Technology, Volume I, Fourth Edition, 1993, pages 572-619, incorporated herein by reference.

Emulsifiers which can be used are all those which are compatible with the system of the invention. In the case of a W/O emulsion, use may be made, for example, of a silicone emulsifier such as cetyl dimethicone copolyol and in the case of an O/W emulsion, use may be made, for example, of one or more fatty acid esters, such as glyceryl stearate, polyethylene glycol stearate containing 40 oxyethylene groups (CTFA name: PEG-40 stearate), sorbitan tristearate and sodium stearate. Other emulsifiers that may be used in the present composition are disclosed in Kirk-Othmer Encyclopedia of Chemical Technology, Volume I, Fourth Edition, 1993, pages 572-619, incorporated herein by reference.

As hydrophilic gelling agents which can be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays such as bentones, and metal salts of fatty acids such as aluminium stearates.

The compositions according to the invention are prepared according to techniques which are well known to those skilled in the art.

In all of the text hereinabove and hereinbelow, the percentages are given on a weight basis except where otherwise mentioned.

Having generally described this invention, a further understanding can be obtained by reference to certain specific examples which are provided herein for purposes of illustration only, and are not intended to be limiting unless otherwise specified.

The names of the compounds are given, depending on the case, as the CTFA name or as the chemical name.

### EXAMPLES

### Example 1

### W/O Emulsion

| | | |
|---|---|---|
| acetylated glycol stearate/tristearin | | 0.12% |
| (Unitwix from the company Guardian) | | |
| magnesium sulphate | | 0.175% |
| polyacrylamide/C13-C14 | | 1% |
| isoparaffin/laureth-7 (Sepigel 305 from | | |
| the company SEPPIC) | | |
| cetyl dimethicone copolyol (Abil EM 90 | | 0.75% |
| from the company Goldschmidt) | | |
| silicone oil | | 12% |
| 1,2-octanediol | | 0.25% |
| sodium capryl lactyl lactylate | | 1% |
| water | qs | 100% |

### Comparative Example 1

The composition is identical to that of Example 1, but without sodium capryl lactyl lactylate.

### Comparative Example 1′

The composition is identical to that of Example 1, but without 1,2-octanediol.

### Example 2

### W/O Emulsion

| | | |
|---|---|---|
| acetylated glycol stearate/tristearin | | 0.12% |
| (Unitwix from the company Guardian) | | |
| magnesium sulphate | | 0.175% |
| polyacrylamide/C13-C14 | | 1% |
| isoparaffin/laureth-7 (Sepigel 305 from | | |
| the company SEPPIC) | | |
| cetyl dimethicone copolyol (Abil EM 90 | | 0.75% |
| from the company Goldschmidt) | | |
| silicone oil | | 12% |
| 1,2-octanediol | | 0.25% |
| Hinokitiol | | 0.01% |
| water | qs | 100% |

### Comparative Example 2

The composition is identical lo that of Example 2, but without 1,2-octanediol.

### Example 3

### W/O Emulsion

| | | |
|---|---|---|
| acetylated glycol stearate/tristearin | | 0.12% |
| (Unitwix from the company Guardian) | | |
| magnesium sulphate | | 0.175% |
| polyacrylamide/C13-C14 | | 1% |
| isoparaffin/laureth-7 (Sepigel 305 from | | |
| the company SEPPIC) | | |
| cetyl dimethicone copolyol (Acil EM 90 | | 0.75% |
| from the company Goldschmidt) | | |
| silicone oil | | 12% |
| 1,2-pentanediol | | 3% |
| sodium capryl lactyl lactylate | | 1% |
| water | qs | 100% |

### Comparative Example 3

The composition is identical to that of Example 3, but without sodium capryl lactyl lactylate.

### Comparative Example 3′

The composition is identical to that of Example 3, but without 1,2-pentanediol.

### Example 4

### W/O Emulsion

| | | |
|---|---|---|
| acetylated glycol stearate/tristearin | | 0.12% |
| (Unitwix from the company Guardian) | | |
| magnesium sulphate | | 0.175% |
| polyacrylamide/C13-C14 | | 1% |
| isoparaffin/laureth-7 (Sepigel 305 from | | |
| the company SEPPIC) | | |
| cetyl dimethicone copolyol (Abil EM 90 | | 0.75% |
| from the company Goldschmidt) | | |
| silicone oil | | 12% |
| 1,2-pentanediol | | 3% |
| Hinokitiol | | 0.01% |
| water | qs | 100% |

### Comparative Example 4

The composition is identical co that of Example 4, but without 1,2-pentanediol.

### Example 5

### Oil-in-water (O/W) Emulsion

| | | |
|---|---|---|
| sorbitan tristearate | | 0.9% |
| liquid petroleum jelly | | 4% |
| isoparaffin | | 10.5% |
| cyclomethicone | | 5% |
| PEG-40 stearate | | 2% |
| cetyl alcohol | | 4% |
| glyceryl stearate (Tegin from the | | 3% |
| company Goldschmidt) | | |
| sodium stearate | | 1.2% |
| 1,2-octanediol | | 0.25% |
| sodium capryl lactyl lactylate | | 1% |
| water | qs | 100% |

### Comparative Example 5

The composition is identical to that of Example 5, but without sodium capryl lactyl lactylate.

### Comparative Example 5′

The composition is identical to that of Example 5, but without 1,2-octanediol.

### Example 6

### Oil-in-water (O/W) Emulsion

| | | |
|---|---|---|
| sorbitan tristearate | | 0.9% |
| liquid petroleum jelly | | 4% |
| isoparaffin | | 10.5% |
| cyclomethicone | | 5% |
| PEG-40 stearate | | 2% |
| cetyl alcohol | | 4% |
| glyceryl stearate (Tegin from the | | 3% |
| company Goldschmidt) | | |
| sodium stearate | | 1.2% |
| 1,2-octanediol | | 0.25% |
| Hinokitiol | | 0.01% |
| water | qs | 100% |

### Comparative Example 6

The composition is identical to that of Example 6, but without 1,2-octanediol.

### Example 7

### O/W Emulsion

| | | |
|---|---|---|
| sorbitan tristearate | | 0.9% |
| liquid petroleum jelly | | 4% |
| isoparaffin | | 10.5% |
| cyclomethicone | | 5% |
| PEG-40 stearate | | 2% |
| cetyl alcohol | | 4% |
| glyceryl stearate (Tegin from the | | 3% |
| company Goldschmidt) | | |
| sodium stearate | | 1.2% |
| 1,2-pentanediol | | 3% |
| sodium capryl lactyl lactylate | | 1% |
| water | qs | 100% |

### Comparative Example 7

The composition is identical to that of Example 7, but without sodium capryl lactyl lactylate.

### Comparative Example 7′

The composition is identical to that of Example 7, but without 1,2-pentanediol.

### Example 8

### O/W Emulsion

| | | |
|---|---|---|
| sorbitan tristearate | | 0.9% |
| liquid petroleum jelly | | 4% |
| isoparaffin | | 10.5% |
| cyclomethicone | | 5% |
| PEG-40 stearate | | 2% |
| cetyl alcohol | | 4% |
| glyceryl stearate (Tegin from the | | 3% |
| company Goldschmidt) | | |
| sodium stearate | | 1.2% |
| 1,2-pentanediol | | 3% |
| Hinokitiol | | 0.01% |
| water | qs | 100% |

### Comparative Example 8

The composition is identical to that of Example 8, but without 1,2-pentanediol.

Demonstration of the Antimicrobial Activity of the Aystem Claimed

The following test demonstrates the antimicrobial activity of the system according to the invention.

1. Materials

a) Microorganisms

Bacterium: Enterococcus faecalis (American Type Culture Collection No. 33186), cultured on soybean Trypticase Yeast: Candida albicans (American Type Culture Collection No. 10231), cultured on Sabouraud.

b) Culture media

Inoculum preparation: tryptone-salt diluent

Counting: Eugon LT100 agar broth

c) Apparatus

Standard equipment of a microbiology laboratory.

2. Method

1. On the evening before the test, each microbial strain is plated out on the appropriate agar medium and incubated in an oven at 35° C.

2. On the day of the test, 20 grams of the product are weighed out in a sterile pill box. Inoculation is carried out at a concentration of 1% using a microbial suspension with a titre of 108 microorganisms per ml: a concentration of 106 is obtained in the product. The contaminated sample is incubated in an oven at 22° C.

3. After incubation for 7 days, 1 gram of product is taken and tenfold dilutions are carried out in Eugon LT100 broth, and these dilutions are plated out on the surface of Petri dishes containing Eugon LT100 agar. The dishes are incubated at 35° C.

4. The colonies are counted on the dishes containing more than 20 and less than 200 colonies.

3. Results

The protocol described above was applied to the compositions of Examples 1 to 8 containing the system of the invention and to the compositions of the comparative examples, as well as to a composition containing none of the compounds of the system of the invention (placebo).

The results obtained are given in the tables below. They are expressed as a number of microorganisms per gram of preparation, remaining in the product after 7 days of contact.

These tests demonstrate the existence of synergism between the two polyols tested and green tea, sodium capryl lactyl lactylate and Hinokitiol. The simultaneous use of a polyol and of one of the three abovementioned compounds in a cosmetic formulation makes it possible to obtain a protection against bacteria and fungi without encountering the skin tolerance problems associated with the use of conventional chemical preserving agents or a high proportion of polyols.

TABLE 1

| | | | Com- | Com- | | Com- |
|---|---|---|---|---|---|---|
| Micro- | | | parative | parative | | parative |
| organism | Placebo | Ex. 1 | Ex. 1 | Ex. 1′ | Ex. 2 | Ex. 2 |
| Enterococcus | 10⁶ | <200 | 10⁴ | 10³ | 10⁴ | 10⁵ |
| faecalis | | | | | | |
| Candida | 10⁶ | <200 | 10⁵ | 10⁶ | <200 | 10⁶ |
| albicans | | | | | | |

TABLE 2

| | | | Com- | Com- | | |
|---|---|---|---|---|---|---|
| Micro- | | | parative | parative | | Comparative |
| organism | Placebo | Ex. 3 | Ex. 3 | Ex. 31′ | Ex. 4 | Ex. 4 |
| Enterococcus | 10⁶ | <200 | 10⁵ | 10³ | <200 | 10⁵ |
| faecalis | | | | | | |
| Candida | 10⁶ | <200 | 10⁶ | 10⁶ | <200 | 10⁶ |
| albicans | | | | | | |

TABLE 3

| | | | | Com- | | Com- |
|---|---|---|---|---|---|---|
| Micro- | | | Comparative | parative | | parative |
| organism | Placebo | Ex. 5 | Ex. 5 | Ex. 5′ | Ex. 6 | Ex. 6 |
| Enterococcus | 10⁶ | <200 | 3.7 × 10⁴ | 10⁴ | <200 | 10⁶ |
| faecalis | | | | | | |
| Candida | 10⁶ | <200 | 10⁶ | 10⁶ | <200 | 10⁵ |
| albicans | | | | | | |

TABLE 4

| | | | | Com- | | Com- |
|---|---|---|---|---|---|---|
| Micro- | | | Comparative | parative | | parative |
| organism | Placebo | Ex. 7 | Ex. 7 | Ex. 7′ | Ex. 8 | Ex. 8 |
| Enterococcus | 10⁶ | <200 | 2.3 × 10⁵ | 10⁴ | <200 | 10⁶ |
| faecalis | | | | | | |
| Candida | 10⁶ | <200 | 10⁶ | 10⁶ | <200 | 10⁵ |
| albicans | | | | | | |

These tables show synergism between the polyol (1,2-octanediol or 1,2-pentanediol) and the combined compound in all the cases for resistance to Candida albicans and in virtually all cases for resistance to Enterococcus faecalis.

Moreover, Table 5 below shows that sodium capryl lactyl lactylate shows synergism with the polyols claimed and not the others, and in particular not with butylene glycol (or butanediol):

TABLE 5

| | | | | | Sodium capryl | Sodium capryl | Sodium capryl |
|---|---|---|---|---|---|---|---|
| | Sodium capryl | | | | lactyl lactylate | lactyl lactylate | lactyl lactylate |
| | lactyl lactylate | Octanediol | pentanediol | Butanediol | (2%) + octanediol | (1%) + pentanediol | (1%) + butanediol |
| Microorganism | (1%) | (0.25%) | (3%) | (3%) | (0.25%) | (3%) | (3%) |
| Enterococcus | 1.5 × 10⁴ | 3.7 × 10⁵ | 2.3 × 10⁵ | 4.0 × 10⁵ | <200 | <200 | 6.8 × 10³ |
| faecalis | | | | | | | |

In microbiology, two values are considered as significantly different when their difference is greater than 0.5 Log. Thus, 6.8×10³ is not significantly different from 1.5×10⁴ and there is thus no synergism between sodium capryl lactyl lactylate and butanediol.

Obviously, numerous modifications and variations of the present invention are possible in light of the above teachings. It is therefore to be understood that within the scope of the appended claims, the invention may be practiced otherwise than as specifically described herein.

The priority document FR 9814077 filed Nov. 9, 1998 is hereby incorporated by reference

1. A antimicrobial agent comprising:
(I) at least one polyol selected from the group consisting of polyols comprising 4 to 8 carbon atoms, mono (C₃-C₉) alkyl, (C₃-C₉) alkenyl glyceryl ethers; and
(II) 2-hydroxy-4-(1-methylethyl)cyclohepta-2,4,6-trien-1-one.

2. The antimicrobial agent of claim 1, wherein the polyol is selected from the group consisting of 1,2-octanediol, 1,2-pentanediol, isoprene glycol, sorbitol, octoxyglycerol, 2-[(heptyl)oxy]-1,2-propanediol, 3-[(octyl)oxy]-1,2-propanediol, 3-[(allyl)oxy]-1,2-propanediol, and mixtures thereof.

3. The antimicrobial agent of claim 1, wherein the weight ratio of the polyol (I) to the compound (II) is from 0.1 to 20,000.

4. A composition comprising, the antimicrobial agent of claim 1 in a physiological acceptable support.

5. The composition of claim 4, wherein the polyol of the antimicrobial agent is from 0.01 to 20% by weight of the total weight of the composition.

6. The composition of claim 4, wherein the compound (II) of the antimicrobial agent is from 0.001 to 10% by weight of the total weight of the composition.

7. The composition of claim 4, wherein the composition is a topical composition

8. The composition of claim 7, wherein the composition is a cosmetic or dermatological composition.

9. The composition of claim 4, wherein the composition is an emulsion.

10. A method of preventing microbial growth in a composition, comprising admixing the antimicrobial agent of claim 1 in said composition.

11. A antimicrobial agent comprising
(I) at least one polyol selected from the group consisting of 1,2-octanediol, 1,2-pentanediol, sorbitol, mono (C₃-C₉) alkyl, and (C₃-Cg) alkenyl glyceryl ethers; and
(II) sodium capryl lactyl lactylate.

12. The antimicrobial agent of claim 11, wherein the weight ratio of the polyol (I) to the compound (II) is from 0.1 to 20,000.

13. A composition comprising, the antimicrobial agent of claim 11 in a physiological acceptable support.

14. The composition of claim 13, wherein the polyol of the antimicrobial agent is from 0.01 to 20% by weight of the total weight of the composition.

15. The composition of claim 13, wherein the compound (II) of the antimicrobial is from 0.001 to 10% by weight of the total weight of the composition.

16. The composition of claim 13, wherein the composition is a topical composition

17. The composition of claim 16, wherein the composition is a cosmetic or dermatological composition.

18. The composition of claim 13, wherein the composition is an emulsion.

19. A method of preventing microbial growth in a composition, comprising admixing the antimicrobial agent of claim 11 in said composition.
